(19) United States
(12) Patent Application Publication
(10) Pub. No.: US 2024/0052007 A1
(43) Pub. Date: Feb. 15, 2024

(54) Fusion protein containing human interleukin-10 and Fc fragment and medical use thereof

(71) Applicant: BEIJING DONGFANG BIOTECH CO., LTD., Beijing (CN)

(72) Inventors: Yi BAI, Beijing (CN); Wen ZHANG, Beijing (CN); Jianhua ZHOU, Beijing (CN)

(21) Appl. No.: 17/766,231

(22) PCT Filed: Sep. 24, 2020
(86) PCT No.: PCT/CN2020/117341

§ 371 Date: Apr. 2, 2022

(30) Foreign Application Priority Data
Oct. 8, 2019 (CN) 201910947766.3

(51) Int. Cl. C07K 14/54; A61P 35/00; C12N 15/85

(52) U.S. Cl. CPC C07K 14/5428; A61P 35/00; C12N 15/85; C07K 2319/30; A61K 38/00

(57) ABSTRACT

Provided are a fusion protein and a medical use thereof. The fusion protein is prepared by fusing a human interleukin-10 (IL-10) with an Fc fragment of an immunoglobulin IgG via a linker peptide. The Fc fragment is the Fc fragment of human IgGI, human IgG2 and human IgG4, and the human interleukin-10 includes an amino acid sequence as shown in SEQ ID NO: 1. The fusion protein may be used for the treatment of immunological diseases and cancers, and prolongs the in vivo half-life of human IL-10 and increases the in vivo stability of human IL-10.

### CROSS-REFERENCE TO RELATED APPLICATION

The present application claims priority to Chinese patent application No. 201910947766.3 filed on Oct. 8, 2019. the disclosure of which is hereby incorporated by reference in its entirety.

### SEQUENCE LISTING

The instant application contains a Sequence Listing which has submitted electronically in ASCII format and is hereby incorporated by reference in its entirety. Said ASCII copy is named PN174773_SEQ_LIST.txt and is 14.306 bytes in size. The sequence listing contains 16 sequences. wherein SEQ ID Nos:1-10 are identical in substance to the sequences disclosed in the PCT application PCT/CN2020/117341 filed on Sep. 24, 2020.SEQ ID Nos:11-16 are newly incorporated based on the sequences [GlyGlyGlyGlyX]ₙ, X is Ser or Ala, and n is an integer of 1-6, so no new matter is included.

### TECHNICAL FIELD

The present disclosure relates to the technical field of biomedicines, in particular to a fusion protein containing human interleukin-10 and Fc fragment and a medical use thereof.

### BACKGROUND

A human interleukin-10 (IL-10) gene is located on 1q31-32 with a full length of 5.1 kb and contains 5 exons. The IL-10 gene consists of 178 amino acids. and 75% of an amino acid sequence of the human and mouse IL-10 genes is identical. The human IL-10 is a 35 kD dimer composed of two monomers in the form of a non-covalent bond, and there are two disulfide bonds within the monomer to maintain the structure and biological activity thereof. At present, it is known that all lymphocytes may synthesize IL-10, and the most important source in vivo is mainly a mononuclear macrophage and a T helper cell. In addition, a dendritic cell, a B cell, a NK cell, a cytotoxic T cell, and a mast cell as well as neutrophil and eosinophil may also synthesize the IL-10 gene.

Over the years, the understanding of people to IL-10 is mainly focused on immunosuppression. It is believed that IL-10 may directly inhibit the proliferation and migration ability of an effector T cell and down-regulate the production of a related cytokine, and play an important role in inducing immune escape of a tumor. In recent years, researches repeatedly show that IL-10 has the immune activation, and the immune activation thereof plays a crucial role in tumor suppression. It is found from the researches by Mumm et al. that pegylated IL-10 has a rejection effect on a transplanted tumor and increases the expression of granzyme B and IFN-γ. IL-10 is a naturally existing immune growth factor in a human body that may stimulate the survival, amplification and killing potential of a special leukocyte in an immune system called as a CD8+T cell. The CD8+T cell may recognize and kill cancer cells. IL-10 activates phosphorylated STAT1 and STAT3 in the CD8+T cell, thereby the proliferation of the CD8+T cell and the expression of IFN-γ, cytotoxic protein perforin and granular protease are induced: IFN-γ may induce the expression of an MHC-I antigen molecule in the tumor cell and the mononuclear macrophage, and assist the CD8+T cell to kill most of the antigen-specific tumor cells; and the activation of a T cell receptor (TCR) in the CD8+T cell may effectively induce an anti-apoptotic signal and a cell proliferation signal, and the survival and amplification of the CD8+T cell are expected to improve the prognosis and survival rate of patients.

The in vivo half-life of recombinant human IL-10 is only 2 hours, and it may be cleared quickly. this seriously affects an application thereof in disease treatment. In order to overcome a problem that the half-life of the recombinant human IL-10 is short, some research institutions currently use a PEGylation modification method to prolong its half-life in vivo, but there are many PEGylation modification sites, so products of the human IL-10 after PEGylation modification are not uniform, this brings great difficulty to the production process and quality control. Therefore, there is an urgent need to develop a human interleukin 10-Fc fusion protein that may effectively prolong the half-life of the recombinant human IL-10. may obtain a stable and uniform product, and is convenient for the process production and quality control, and a medical use thereof.

### SUMMARY

In order to solve problems in an existing technology that the half-life of human IL-10 is short, products of the human IL-10 after PEGylation modification are not uniform and the like, through a genetic engineering technology, the present disclosure provides a human interleukin 10-Fc fusion protein that fuses human IL-10 and an immunoglobulin Fc fragment together, retains the biological activity of IL-10, and greatly prolongs the half-life of IL-10 in an organism body, and a medical use thereof.

A specific technical scheme of the present disclosure is as follows.

The present disclosure provides a fusion protein containing a human interleukin-10 and an Fc fragment, the fusion protein is formed by fusing the human interleukin-10 with the Fc fragment of an immunoglobulin IgG via a linker peptide, herein, the Fc fragment is an Fc fragment of human IgG1, human IgG2 and human IgG4.

The Fc fragment of the human IgG4 includes an amino acid sequence shown in SEQ ID No: 6 (or the amino acid sequence of the Fc fragment of the human IgG4 is shown in SEQ ID No: 6).

The human interleukin-10 includes an amino acid sequence shown in SEQ ID No: 1.

The Fc fragment of the human IgG2 includes an amino acid sequence shown in SEQ ID No: 5 (or the amino acid sequence of the Fc fragment of the human IgG2 is shown in SEQ ID No: 5).

In the fusion protein of the present disclosure, the human interleukin-10, the linker peptide and the Fc fragment of the immunoglobulin IgG are sequentially linked in the manner of “N-terminal” to “C-terminal”.

The present disclosure fuses the human interleukin-10 with the immunoglobulin Fc fragment, and it not only retains the biological activity of the human interleukin-10, but also effectively prolongs the half-life through the immunoglobulin Fc fragment and overcomes a defect that the half-life of the human interleukin-10 is short. The Fc fragment of the present disclosure is derived from human IgG1, IgG2 or IgG4, herein IgG2 and IgG4 use a wild-type sequence, among four subtypes of human IgG, the antibody-dependent cell-mediated cytotoxicity (ADCC) and the complement-dependent cytotoxicity (CDC) of the IgG1 and IgG3 subtypes are relatively strong, and the ADCC and CDC of the IgG2 and IgG4 subtypes are relatively weak. The fusion protein of the present disclosure does not require the ADCC and CDC, and on the contrary, the ADCC and CDC may bring some unnecessary side effects. Therefore, according to a preferred embodiment of the present disclosure, the Fc fragment of IgG2 and IgG4 uses a wild-type amino acid sequence. In addition, the present disclosure not only guarantees the stability of the macromolecular fusion protein, but also guarantees that a uniform fusion protein product may be obtained by linking the linker peptide to the Fc fragment of the immunoglobulin IgG, and it is convenient for production and quality control.

Preferably, the Fc fragment is an Fc portion of human IgG1. The Fc portion of the human IgG1 includes an amino acid sequence shown in SEQ ID No: 2 or SEQ ID No: 3, or the amino acid sequence of the Fc portion of the human IgG1 is shown in SEQ ID No: 2 or SEQ ID No: 3.

Preferably, the Fc fragment includes an amino acid sequence shown in SEQ ID No: 2 or SEQ ID No: 3.

Further, a general formula of the linker peptide is [GlyGlyGlyGlyX]ₙ.

Herein, X is Ser or Ala, and n is an integer of 1-6(corresponding to the SEQ ID Nos: 11-16 of the sequence listing, respectively).

Preferably, X is Ser.

Preferably, n is 6.

The linker peptide structure of the above general formula may further guarantee the biological activity of a drug molecule.

The present disclosure further provides a polynucleotide sequence, and the polynucleotide sequence encodes the amino acid sequence of the fusion protein.

The present disclosure further provides a recombinant deoxyribonucleic acid (DNA) expression vector, and the recombinant DNA expression vector contains the above polynucleotide sequence.

The present disclosure further provides a host cell for transfected with the above recombinant DNA expression vector, and the host cell includes at least one of a prokaryotic cell, a yeast cell and a mammalian cell.

The present disclosure further provides a drug or a pharmaceutical composition, and the drug or the pharmaceutical composition contains the above fusion protein.

The present disclosure further provides an application of the above fusion protein for preparing a drug for preventing and/or treating an immune disease and/or a cancer.

The beneficial effects of the present disclosure are as follows: first, while the biological activity of the human interleukin-10 is retained, the fusion protein provided by the present disclosure prolongs the half-life of the human interleukin-10 in the organism by fusing with the Fc fragment of the immunoglobulin IgG, the stability of the human interleukin-10 in vivo is increased, the tumor growth may be inhibited for a long time, and it is beneficial to the application thereof in disease treatment. Second, from the perspective of purification process and production, the present disclosure adopts a genetic engineering technology to prepare the fusion protein, the human interleukin-10 is fused with the Fc fragment of the immunoglobulin IgG through the linker peptide, the structural stability of the macromolecular fusion protein is improved, it is not easy to decompose, the product uniformity is good. It is convenient for purification, the purity of a purified product is guaranteed, and troubles of production process and quality control brought about by IL-10 PEGylation are overcome.

In addition, the IL-10 fusion protein may be used for the treatment of a related disease, the disease includes the immune disease and/or the cancer, the immune disease includes, but is not limited to, multiple sclerosis, psoriasis, rheumatoid arthritis, Crohn's disease and the like. The cancer includes, but is not limited to, a pancreatic cancer, a non-small cell lung cancer, a melanoma, a prostate cancer, a kidney cancer, a colorectal cancer, a breast cancer and other tumors.

### BRIEF DESCRIPTION OF THE DRAWINGS

FIG. 1 is a molecular structure schematic diagram of a fusion protein of the present disclosure.

FIG. 2 is a human interleukin-10 and a fusion protein expression vector of the present disclosure.

FIG. 3 is a denaturing polyacrylamide gel electrophoresis of the human interleukin-10 and the fusion protein of the present disclosure.

FIG. 4 shows that the human interleukin-10 and the fusion protein of the present disclosure stimulate proliferation of a mouse mast cell MC/9.

FIG. 5 shows killing of a SK-BR-3 tumor cell by the fusion protein of the present disclosure.

FIG. 6 is a pharmacodynamic effect of the fusion protein of the present disclosure in a mouse H1975 tumor model.

FIG. 7 is a diagram showing the in vivo pharmacodynamic effect of the fusion protein of the present disclosure on a humanized xenografted non-small cell lung cancer H1975 tumor model.

### DETAILED DESCRIPTION OF THE EMBODIMENTS

The present disclosure is further described in detail below in combination with the following embodiments.

### Embodiment 1

Embodiment 1 of the present disclosure provides a fusion protein, and the fusion protein is formed by fusing a human interleukin-10 with an Fc fragment of an immunoglobulin IgG via a linker peptide; herein the Fc fragment is an Fc portion of human IgG1: an amino acid sequence of the human interleukin-10 is shown in SEQ ID No: 1; and an amino acid sequence of the Fc fragment is shown in SEQ ID No: 2.

A general formula of the linker peptide is [GlyGlyGlyGlySer]₆, and an amino acid sequence of the linker peptide is shown in SEQ ID No: 4.

Herein, SEQ ID No: 1 (the amino acid sequence of
the human interleukin-10):
SPGQGTQSENSCTHFPGNLPNMLRDLRDAFSRVKTFFQMKDQLDNLLLK
ESLLEDFKGYLGCQALSEMIQFYLEEVMPQAENQDPDIKAHVNSLGENL
KTLRLRLRRCHRFLPCENKSKAVEQVKNAFNKLQEKGIYKAMSEFDIFI
NYIEAYMTMKIRN.
SEQ ID No: 2 (the amino acid sequence of the Fc
fragment):
DKTHTCPPCPAPELEGGPSVFLFPPKPKDTLMISRTPEVTCVVVDVSHE
DPEVKFNWYVDGVEVHNAKTKPREEQYNSTYRVVSVLTVLHQDWLNGKA
YACAVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK.
SEQ ID No: 4 (the amino acid sequence of the
linker peptide):
GGGGSGGGGSGGGGSGGGGGGGGSGGGGS.

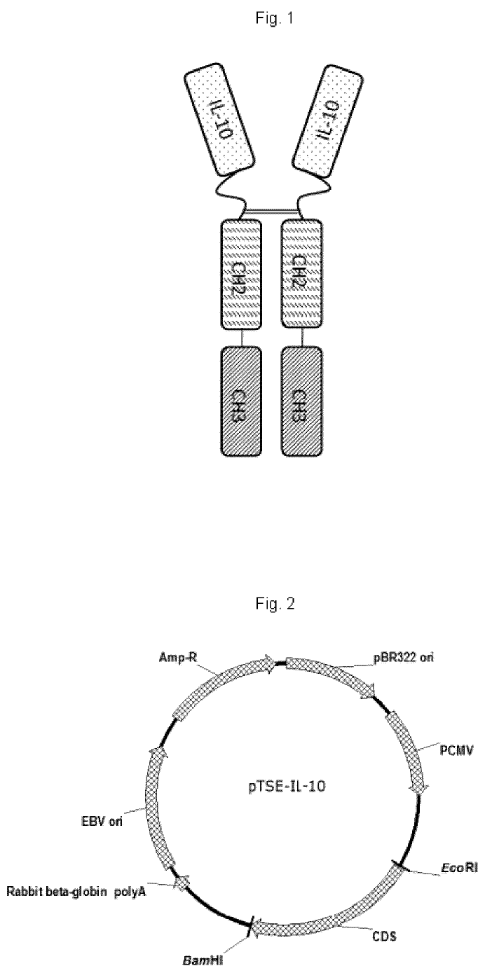

A schematic diagram of a specific configuration of the fusion protein is shown in FIG. 1.

### Embodiment 2

Embodiment 2 of the present disclosure provides a fusion protein, and the fusion protein is formed by fusing a human Interleukin-10 with an Fc fragment of an immunoglobulin IgG via a linker peptide; herein the Fc fragment is an Fc portion of human IgG1: an amino acid sequence of the human interleukin-10 is shown in SEQ ID No: 1; and an amino acid sequence of the Fc fragment Is shown in SEQ ID No: 3.

A general formula of the linker peptide is [GlyGlyGlyGlySer]₆, and an amino acid sequence of the linker peptide is shown in SEQ ID No: 4.

Herein, the amino acid sequences provided by SEQ ID No: 1 and SEQ ID No: 4 are the same as those in Embodiment 1.

SEQ ID No: 3 (the amino acid sequence of the Fc
fragment):
EPKSCDKTHTCPPCPAPELEGGPSVFLFPPKPKDTLMISRTPEVTCVVV
DVSHEDPEVKFNWYVDGVEVHNAKTKPREEQYNSTYRVVSVLTVLHQDW
LNGKAYACAVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

A schematic diagram of a specific configuration of the fusion protein is shown in FIG. 1.

### Embodiment 3

Embodiment 3 of the present disclosure provides a fusion protein, and the fusion protein is formed by fusing a human interleukin-10 with an Fc fragment of an immunoglobulin IgG via a linker peptide; herein the Fc fragment is an Fc portion of human IgG2; an amino acid sequence of the Fc fragment is shown in SEQ ID No: 5; and an amino acid sequence of the human interleukin-10 is shown in SEQ ID No: 1.

A general formula of the linker peptide is [GlyGlyGlyGlySer]₆, and an amino acid sequence of the linker peptide is shown in SEQ ID No: 4.

Herein, the amino acid sequences provided by SEQ ID No: 1 and SEQ ID No: 4 are the same as those in Embodiment 1.

SEQ ID No: 5 (the amino acid sequence of the Fc
fragment):
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPGK.

A schematic diagram of a specific configuration of the fusion protein is shown in FIG. 1.

### Embodiment 4

Embodiment 4 of the present disclosure provides a fusion protein, the fusion protein is formed by fusing a human interleukin-10 with an Fc fragment of an immunoglobulin IgG via a linker peptide; herein the Fc fragment is an Fc portion of human IgG4; an amino acid sequence of the Fc fragment is shown in SEQ ID No: 6; and an amino acid sequence of the human interleukin-10 is shown in SEQ ID No: 1.

A general formula of the linker peptide is [GlyGlyGlyGlySer]₆. and an amino acid sequence of the linker peptide is shown in SEQ ID No: 4.

Herein, the amino acid sequences provided by SEQ ID No: 1 and SEQ ID No: 4 are the same as those in Embodiment 1.

SEQ ID No: 6 (the amino acid sequence of the Fc
fragment):
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A schematic diagram of a specific configuration of the fusion protein is shown in FIG. 1.

### Embodiment 5

Embodiment 5 of the present disclosure provides a fusion protein, and the fusion protein is formed by fusing a human interleukin-10 with an Fc fragment of an immunoglobulin IgG via a linker peptide; herein the Fc fragment is an Fc portion of human IgG2; an amino acid sequence of the Fc fragment is shown in SEQ ID No: 5; and an amino acid sequence of the human interleukin-10 is shown in SEQ ID No: 1.

A general formula of the linker peptide is [GlyGlyGlyGlySer]s, and an amino acid sequence of the linker peptide is shown in SEQ ID No: 7.

Herein, the amino acid sequence provided by SEQ ID No: 1 is the same as that in Embodiment 1; and the amino acid sequence provided by SEQ ID No: 5 is the same as that in Embodiment 3.

SEQ ID No: 7 (the amino acid sequence of the
linker peptide):
GGGGSGGGGSGGGGSGGGGSGGGGS.

A schematic diagram of a specific configuration of the fusion protein is shown in FIG. 1.

### Embodiment 6

Embodiment 6 of the present disclosure provides a fusion protein, and the fusion protein is formed by fusing a human interleukin-10 with an Fc fragment of an immunoglobulin IgG via a linker peptide; herein the Fc fragment is an Fc portion of human IgG4; an amino acid sequence of the Fc fragment is shown in SEQ ID No: 6; and an amino acid sequence of the human interleukin-10 is shown in SEQ ID No: 1.

A general formula of the linker peptide is [GlyGlyGlyGlyAla]₄, and an amino acid sequence of the linker peptide is shown in SEQ ID No: 8.

Herein, the amino acid sequence provided by SEQ ID No: 1 is the same as that in Embodiment 1; and the amino acid sequence provided by SEQ ID No: 6 is the same as that in Embodiment 4.

SEQ ID No: 8 (the amino acid sequence of the
linker peptide):
GGGGAGGGGAGGGGGGGGA.

A schematic diagram of a specific configuration of the fusion protein is shown in FIG. 1.

### Embodiment 7

Embodiment 7 of the present disclosure provides a fusion protein, and the fusion protein is formed by fusing a human interleukin-10 with an Fc fragment of an immunoglobulin IgG via a linker peptide; herein the Fc fragment is an Fc portion of human IgG1; an amino acid sequence of the human interleukin-10 is shown in SEQ ID No: 1: and an amino acid sequence of the Fc fragment is shown in SEQ ID No: 2.

A general formula of the linker peptide is [GlyGlyGlyGlySer]₃, and an amino acid sequence of the linker peptide is shown in SEQ ID No: 9.

Herein, the amino acid sequences provided by SEQ ID No: 1 and SEQ ID No: 2 are the same as those in Embodiment 1.

SEQ ID No: 9 (the amino acid sequence of the
linker peptide):
GGGGSGGGGSGGGGS.

A schematic diagram of a specific configuration of the fusion protein is shown in FIG. 1.

### Embodiment 8

Embodiment 8 of the present disclosure provides a fusion protein, and the fusion protein is formed by fusing a human interleukin-10 with an Fc fragment of an immunoglobulin IgG via a linker peptide: herein the Fc fragment is an Fc portion of human IgG2; the amino acid sequence of the human interleukin-10 is shown in SEQ ID No: 1; and the amino acid sequence of the Fc fragment is shown in SEQ ID No: 5.

A general formula of the linker peptide is [GlyGlyGlyGlyAla]₃, and an amino acid sequence of the linker peptide is shown in SEQ ID No: 10.

Herein, the amino acid sequence provided by SEQ ID No: 1 is the same as that in Embodiment 1: and the amino acid sequence provided by SEQ ID No: 5 is the same as that in Embodiment 3.

SEQ ID No: 10 (the amino acid sequence of the
linker peptide):
GGGGAGGGGAGGGGA.

A schematic diagram of a specific configuration of the fusion protein is shown in FIG. 1.

### Embodiment 9

Embodiment 9 of the present disclosure provides a polynucleotide sequence, and the polynucleotide sequence encodes the amino acid sequence of the fusion protein provided in any one of Embodiments 1-8.

### Embodiment 10

Embodiment 10 of the present disclosure provides a recombinant DNA expression vector, and the recombinant DNA expression vector contains the polynucleotide sequence provided in Embodiment 9.

### Embodiment 11

Embodiment 11 of the present disclosure provides a host cell for transfected with the recombinant DNA expression vector provided in Embodiment 10, and the host cell includes a prokaryotic cell, a yeast cell and a mammalian cell.

### Embodiment 12

Embodiment 12 of the present disclosure provides a drug or a pharmaceutical composition. and the drug or the pharmaceutical composition contains the fusion protein provided in any one of Embodiments 1-8.

### Embodiment 13

Embodiment 13 of the present disclosure provides an application of a fusion protein for preparing a drug for treatment of an immune disease and a cancer, herein the immune disease includes, but is not limited to, multiple sclerosis, psoriasis, rheumatoid arthritis, Crohn's disease and the like; and the cancer includes, but is not limited to, a pancreatic cancer, a non-small cell lung cancer, a melanoma, a prostate cancer, a kidney cancer, a colorectal cancer, a breast cancer and other tumors.

### Example 1

This Example is used to describe the construction of a human interleukin-10 and a fusion protein expression vector of the present disclosure.

According to Embodiments 1-8 and with reference to the molecular configuration schematic diagram shown in FIG. 1, pTSE (for the preparation process of a pTSE vector, reference to

Paragraph on Page 3 of the description of CN103525868A and Embodiment 1) is selected as an expression vector, and a gene encoding a fusion protein is synthesized by Nanjing Genscript Biotechnology Co., Ltd. During the gene synthesis, EcoR1 and BamHI restriction sites are introduced on both sides of the synthesized gene, and then EcoR1 and BamHI double enzyme digestion is performed on the pTSE expression vector and the synthesized gene encoding the fusion protein, enzyme digestion products of the pTSE expression vector and the gene encoding the fusion protein are subjected to agarose gel electrophoresis and target fragment recovery, and finally a recovered target fragment is connected to the pTSE expression vector respectively, and transformed into a TOP competent cell (HuitianDongfang, product number HT702-03), the gene expression vector (as shown in FIG. 2) is obtained after sequencing is correct, a human interleukin-10 expression plasmid is named as r1L-10, and according to Table 1, the expression plasmids of the fusion proteins of Embodiments 1-8 are named as: IL-10-Fc-A, IL-10-Fc-B, IL-10-Fc-C, IL-10-Fc-D, IL-10-Fc-E, IL-10-Fc-F, IL-10-Fc-G, IL-10-Fc-H.

### Example 2

This Example is used to describe the expression and purification of a human interleukin-10 and a fusion protein of the present disclosure.

### I) Acquisition of Human Interleukin-10 and Fusion Protein Expression Plasmid.

An endotoxin-free large-scale extraction kit (purchased from Kangwei Century Biotechnology Co., Ltd., CW2104) is used to perform plasmid large-scale extraction, and the specific operation steps are as follows:
(1) 200 μl of activated bacterial solution is taken and placed in a 500 ml shake flask (Luria-Bertani (LB) medium containing 200 ml adenosine monophosphate (amp+)), and it is cultured overnight on a shaker at 37° C. and 220 rpm.
(2) 200 ml of the overnight cultured bacterial solution is taken, and added to a centrifuge tube, it is centrifuged at 7000 rpm for 5 minutes to collect bacteria, and all supernatants are removed as much as possible.
(3) 12.5 ml of Buffer P1 (RNase A is added already) is added to the centrifuge tube with a bacterial precipitate left, and it is adequately mixed uniformly with a vortex shaker, to suspend the bacterial precipitate.
(4) 12.5 ml of Buffer P2 is added to the centrifuge tube, it is inverted up and down gently and mixed uniformly for 8-10 times, to fully lyse the bacteria, and it is placed at a room temperature for 3-5 minutes, until the solution becomes clear and viscous.
(5) 12.5 ml of Buffer E3 is added to the centrifuge tube, and it is inverted up and down immediately and mixed uniformly for 8-10 times. At this time, a white flocculent precipitate appears. and it is placed at the room temperature for 5 minutes; it is centrifuged at 7000rpm for 15 minutes, all supernatants are poured into an endotoxin filter (Endo-Remover FQ), a push handle (Plungers) is slowly pushed to filter, and filtrate is collected in a clean 50 ml centrifuge tube (self-provided).
(6) 10 ml of isopropanol which is 0.3 times greater than the filtrate volume is added to the filtrate. and it is inverted up and down and mixed uniformly.
(7) Column equilibration: 2 ml of Buffer PS is added to an adsorption column (Spin Columns DQ) that is already placed in a collection tube, it is centrifuged at 7000 rpm for 2 minutes, waste liquid in the collection tube is poured off, and the adsorption column is placed in the collection tube again.
(8) In the step 6, the mixed solution of the filtrate and isopropanol is transferred to the well-balanced adsorption column (placed in the collection tube already).
(9) It is centrifuged at 7000 rpm for 2 minutes, the waste liquid in the collection tube is poured off, and the adsorption column is placed in the collection tube again.
(10) 10 ml of Buffer PW (absolute ethanol is added already) is added to the adsorption column, it is centrifuged at 7000 rpm for 2 minutes. and the waste liquid in the collection tube is poured off.
(11) The step (10) is repeated once.
(12) The adsorption column is placed the collection tube again, it is centrifuged at 7000 rpm for 5 minutes, the waste liquid is poured off, and the adsorption column is placed at the room temperature for several minutes, to thoroughly dry residual rinsing solution in the adsorption column.
(13) The adsorption column is placed in a new centrifuge tube, 1 ml of endotoxin-free water is added to a middle part of the adsorption column, it is placed at the room temperature for 2-5 minutes, and centrifuged at 7000 rpm for 5 minutes, and plasmid solution is collected into the centrifuge tube; and after the concentration is measured, the plasmid is stored at −20° C.

### II). Transient Transfection of Human Interleukin-10 and Fusion Protein Expression Plasmid.

A human embryonic kidney cell (HEK293 suspension cell, purchased from the Institute of Basic Medicine, Chinese Academy of Medical Sciences, article number GNHu43) is cultured in a FreeStyle 293 Expression Medium (Gibco), and the cells are passaged every one to two days. The initial cell density after passage is maintained at 0.2-0.6×10⁶ cells/ml, the cell culture volume is 15-35% of the volume of the shaker flask, and a cell culture flask is placed on a shaker (shaker revolution speed: 135 rpm. temperature: 37° C., and CO₂: 5%) for culture. One day before transfection, HEK293 cells in a logarithmic growth phase and in a good growth state are passaged to a cell density of 0.5×10⁶ cells/ml, it is placed on the shaker (135 rpm, 37° C. and 5% CO₂) and cultured ovemight, and transfection is performed on the next day.

Before the transfection. 1×10⁶ cells/ml of prepared cell suspension is cultured on the shaker (135 rpm. 39° C., 5% CO₂) for 2 h. During the transfection, 9 plasmids (1 μg/ml of the final concentration) obtained in the above step 1), and polyethyleneimine PEI (2 μg/ml of the final concentration) are sequentially added, mixed uniformly, and co-transfected into the HEK293 suspension cells together, and then it is placed on the shaker (135 rpm. 39° C., and 5% CO₂) and cultured for 40 min. The transfected cells are continued to be cultured on the shaker at 135 rpm. 37° C., and 5% CO₂ to express the human interleukin-10 and the fusion protein. The supernatant is harvested by centrifugation after 96 hours of the transfection.

### III). Purification of Human Interleukin-10 and Fusion Protein.

Purification of human interleukin-10: supernatant liquid is filtered with a 0.22 μM filter membrane, and the human interleukin-10 with a His-tag domain is obtained from the expression supernatant by using a Ni column. Equilibration buffer and elution buffer are 50 mMTris-HCl, 0.5M NaCl, 20 mM imidazole, pH 7.6 and 50 mMTris-HCl, 0.5M NaCl, 0.5M imidazole, pH 7.6 respectively. Setting of gradient elution conditions: 100% eluent, 30 min, and 5 ml/min flow rate gradient elution, according to different concentrations of imidazole, protein peaks detected by UV280 are collected, the peak position is marked, and phosphate buffer saline (PBS) is used for liquid concentration.

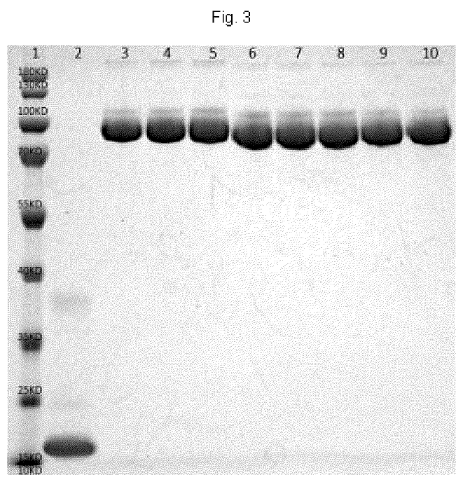

Purification of fusion protein: the supernatant is filtered with the 0.22 μM filter membrane, and the fusion protein with an Fc domain is obtained from the expression supernatant by using a HITraprProtein A affinity chromatography column. Equilibration buffer and elution buffer are 50 mMTris-HCl, 0.15M NaCl, pH 7.0 and 0.1M citric acid-sodium citrate, pH 3.0 respectively. The target fusion protein is obtained by a cation exchange column HiTrap-SPFF, and finally the PBS is used for the liquid concentration. The purified human interleukin-10 and fusion protein are obtained, as shown in FIG. 3, from left to right, they are protein molecular weight Marker, rIL-10, IL-10-Fc-A, IL-10-Fc-B, IL-10-Fc-C, IL-10-Fc-D, IL-10-Fc-E, IL-10-Fc-F, IL-10-Fc-G, IL-10-Fc-H sequentially. and the molecular weight of each band is consistent with the theory.

TABLE 1

| | | Human | | | |
|---|---|---|---|---|---|
| | | interleukin-10 | | Fc amino | Linker peptide |
| | Drug | amino acid | | acid | amino acid |
| Embodiments | molecule | sequence | Fcsubtype | sequence | sequence |
| 1 | IL-10-Fc-A | SEQ ID No: 1 | IgG1 | SEQ ID No: 2 | SEQ ID No: 4 |
| 2 | IL-10-Fc-B | SEQ ID No: 1 | IgG1 | SEQ ID No: 3 | SEQ ID No: 4 |
| 7 | IL-10-Fc-C | SEQ ID No: 1 | IgG1 | SEQ ID No: 2 | SEQ ID No: 9 |
| 3 | IL-10-Fc-D | SEQ ID No: 1 | IgG2 | SEQ ID No: 5 | SEQ ID No: 4 |
| 5 | IL-10-Fc-E | SEQ ID No: 1 | IgG2 | SEQ ID No: 5 | SEQ ID No: 7 |
| 8 | IL-10-Fc-F | SEQ ID No: 1 | IgG2 | SEQ ID No: 5 | SEQ ID No: 10 |
| 4 | IL-10-Fc-G | SEQ ID No: 1 | IgG4 | SEQ ID No: 6 | SEQ ID No: 4 |
| 6 | IL-10-Fc-H | SEQ ID No: 1 | IgG4 | SEQ ID No: 6 | SEQ ID No: 8 |

### Example 3

This Example is used to describe the effect of a fusion protein of the present disclosure in stimulating the proliferation of a mouse mast cell MC/9.

### 1. Experimental Cell

Name: mouse mast cell MC/9
Cell culture medium: RPMI1640 (Gibico, A10491-01)+10% Foetal Bovine Serum (FBS) (VisTech, SE200-ES)
Source: Shanghai Zishi Biotechnology Co., Ltd.
Cell characteristics: the cell expresses an endogenous mouse interleukin-10 (IL-10) receptor (R1 and R2), and in the presence of a mouse interleukin-4 (IL-4) (purchased from Nanjing Genscript Biotechnology Co., Ltd., and article number is Z02996). IL-10 may stimulate the proliferation of a MC/9 mouse mast cell line. However, single mIL-4 or hIL-10 has only very low proliferation stimulating activity.

### 2. Cell Planking and Dosing

The MC/9 cells in a logarithmic growth phase are taken and washed twice with a blank 1640 medium, it is suspended in a 20% FCS-1640 medium, and adjusted to a concentration of 2×10⁵ cells/ml, it is added to a 96-well plate at 1×10⁴ cells/well, a control group and an experimental group are set, and administered separately. The administration dose of the fusion protein in a single-action group is 25 ng/well, and the administration doses of mIL-4 and fusion protein in a combined-action group are 0.05 ng/well and 25 ng/well respectively, and the specific dosing situation is shown below in Table 2.

TABLE 2

Experimental group

| Control group | Single-action group | Combined-action group |
|---|---|---|
| Medium | IL-10-Fc-A | mIL-4 + IL-10-Fc-A |
| | IL-10-Fc-B | mIL-4 + IL-10-Fc-B |
| rIL-10 | IL-10-Fc-C | mIL-4 + IL-10-Fc-C |
| | IL-10-Fc-D | mIL-4 + IL-10-Fc-D |
| mIL-4 | IL-10-Fc-E | mIL-4 + IL-10-Fc-E |
| | IL-10-Fc-F | mIL-4 + IL-10-Fc-F |
| rIL-10 + mIL-4 | IL-10-Fc-G | mIL-4 + IL-10-Fc-G |
| | IL-10-Fc-H | mIL-4 + IL-10-Fc-H |

After culturing for 72 hours in a 37° C. and 5% CO₂ Incubator, CCK-8 detection solution is added, and an optical density (OD) value Is detected at 450 nm after Incubation at 37° C. for 2-4 hours.

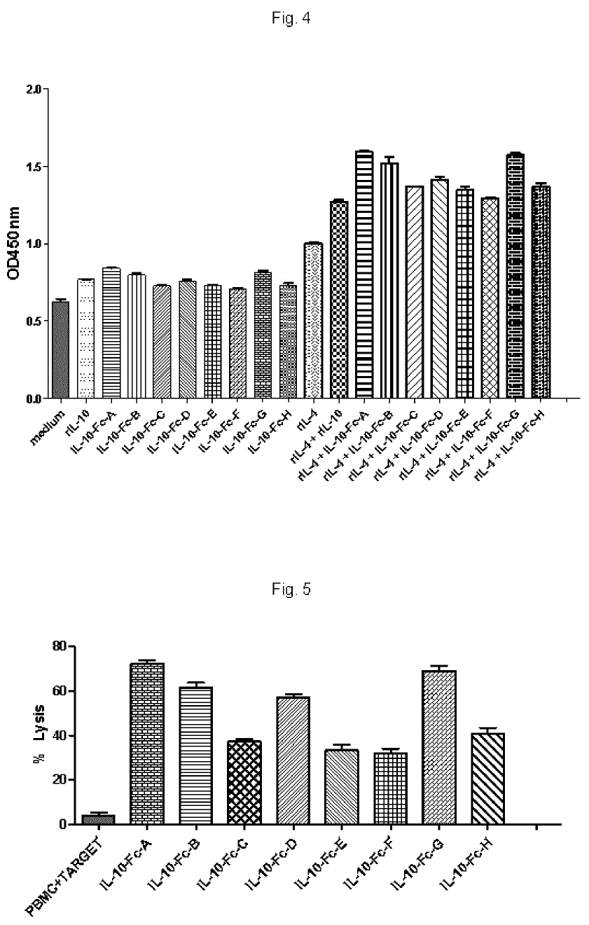

Experimental results are shown in FIG. 4. It is known from the cell proliferation situation that the fusion proteins IL-10-Fc-A, IL-10-Fc-B, IL-10-Fc-C, IL-10-Fc-D, IL-10-Fc-E, IL-10-Fc-F, IL-10-Fc-G, and IL-10-Fc-H provided by the present disclosure retain the biological activity of the human interleukin-10, and the different types of the fusion proteins may stimulate the proliferation of the mouse mast cell MC/9, and through co-stimulation with the mouse Interleukin-4 (IL-4), the fusion protein may significantly stimulate the cell proliferation.

### Example 4

This Example is used to describe the in vitro killing effect of a fusion protein of the present disclosure on a SK-BR-3 tumor cell.

### 1. Target Cell

Name: human breast cancer cell SK-BR-3
Maintenance medium: RPMI1640 (Gibico. A10491-01)+10% FBS (VisTech, SE200-ES)
Experimental medium: RPMI1640 (Gibico, A10491-01)+10% inactivated FBS (VisTech, SE200-ES)
Source: purchased from American Type Culture Collection (ATCC), the number is HTB-30.

### 2. Target Cell Planking and Serum Inactivation

After the SK-BR-3 cells are digested by one day in advance, the cells are resuspended and counted in the maintenance medium, and plated in a flat-bottom 96-well plate at 5×10³ cells/100 μl/well, and it is cultured overnight until the cells are adhered to a wall.

A tube of completely melted FBS is taken and placed in a 60° C. water bath for 40 min. to obtain the inactivated serum.

### 3. Effector Cell-Human Mononuclear Cell (Peripheral Blood Mononuclear Cell (PBMC)) Separation

20 ml of mononuclear cell separation solution is added to a 50 ml tube: collected blood is diluted with whole blood diluent at a ratio of 1:1, after being mixed uniformly, it is slowly spread along an inner wall of a Corning tube at a uniform speed to an upper layer of the separation solution, and the volume of the whole blood after dilution added in each tube is 20 ml; after liquid is added to each tube, it is put in a centrifuge pre-cooled to 22° C. in advance, and it is centrifuged horizontally at 600 g for 15 min (acceleration and deceleration are set to 1): after centrifugation, the centrifuge tube is taken out, and a pipet is used to carefully aspirate a cell layer-mononuclear cells (PBMC) placed in a circular arc shape between the separation solution and the serum, and it is placed in a new 50 ml tube: cell washing solution is add to the cell solution at a ratio of 1:5, it is centrifuged after being adequately mixed uniformly, supernatant is discarded, washing is repeated once, a cell precipitate is collected, and it is resuspended with the RPMI-1640 medium; and the number of the mononuclear cells (PBMC) is adjusted to 2.5×10⁶ cells/ml.

### 4. Drug Dilution and Planking and Dosing

Eight drug molecules (IL-10-Fc-A, IL-10-Fc-B, IL-10-Fc-C, IL-10-Fc-D, IL-10-Fc-E, IL-10-Fc-F, IL-10-Fc-G, and IL-10-Fc-H) obtained in Example 2 are diluted with the experimental medium, so that the action final concentration thereof is 200 μg/ml, and 3 duplicate wells are set.

The growth medium in the 96-well plate is discarded, sterilized PBS is gently washed once, and 100 μl of the experimental medium is added to each well. The PBMC cells with the adjusted number and the diluted drug molecules are successively added to the 96-well plate, and the effect-to-target ratio of PBMC to the target cells is 50:1.

Blank target cells and blank PBMC control group are set, each group contains only the target cells and effector cells respectively, each group has 3 duplicate wells; and at the same time, effector cell/target cell mixed effect wells are set in a total of 6 wells. Herein. 3 wells are set as a maximum killing group, and lysis solution is added in 30 min before detection, to completely lyse and kill the cells; and the remaining 3 wells are a natural killing group (in the natural killing group, IL-10-Fc-A, IL-10-Fc-B, IL-10-Fc-C, IL-10-Fc-D, IL-10-Fc-E, IL-10-Fc-F, IL-10-Fc-G, and IL-10-Fc-H are added respectively), as a control for the killing effect of the fusion protein (TARGET+PBMC). The blank target cells, the blank PBMC and the maximum killing group are all used for cell death rate calculation. After a label is clear, it is placed in a 37° C. cell incubator for incubation.

### 5. Detection and Killing Rate Calculation

After 3 days, the number of the target cells is apparently decreased, the supernatant is taken for lactate dehydrogenase (LDH) detection, and the cell death rate is calculated after OD490 is read with a microplate reader. A calculation formula is as follows:

$$\frac{experimental well - blank target cell - blank PBMC}{maximal killing well - blank target cell - blank PBMC} \times 100 \% = Cell death rate ( \% )$$

Experimental results are shown in FIG. 5. Compared with the control group PBMC+TARGET, the eight fusion proteins provided in examples 1-8 may specifically kill the SK-BR-3 tumor cells, herein IL-10-Fc-A, IL-10-Fc-B, IL-10-Fc-D, and IL-10-Fc-G have the stronger killing ability, and the cell death rates are all 60% or more. The IL-10-Fc-A fusion protein has the strongest killing ability.

### Example 5

This Example is used to describe the efficacy of a fusion protein of the present disclosure in a mouse H1975 tumor model.

1. Experimental animal:
Species strain: MusMusculus, NCG, mouse
Week age: 6-8 weeks
Experimental animal provider: Gempharmatech Co., Ltd.
   2. Cell culture: tumor cells (purchased from ATCC, the article number is CRL-5908) are cultured in an incubator at 37° C. and 5% CO₂ with a RPMI-1640 medium containing inactivated 10% FBS, 100 U/ml penicillin, 100 μg/ml streptomycin and 2 mM glutamine, the cells are passaged in bottles every 3 to 4 days after the cells are fully grown, and the tumor cells in a logarithmic growth phase are used for inoculation of a tumor in vivo.
   3. Inoculation and grouping of tumor cells:

The H1975 cells are washed twice with PBS, and then the tumor cells are resuspended in PBS, to obtain NCl-H1975 human non-small cell lung cancer cells, and the NCl-H1975 human non-small cell lung cancer cells are inoculated into a PBMC humanized NCG mouse subcutaneously, the inoculation amount of cells is 5×10⁶/mouse; and PBMC is derived from normal human peripheral blood, and is inoculated into a tumor-bearing mouse in three days before H1975 cell inoculation at 2×10⁶/mouse. After the tumor inoculation, while the tumor grows to about 100 mm³, it is administered in groups at a total of 6 groups, there are 8 animals in each group, namely a vehicle control group, a rIL-10 group, a IL-10-Fc-A group, a IL-10-Fc-B group, a IL-10-Fc-D group, and a IL-10-Fc-G group (1mg/kg, sc, biw×4 Weeks).

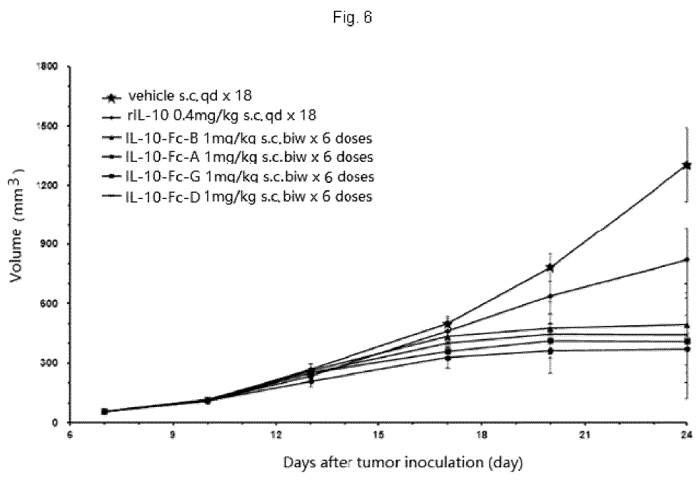

4. Detection Indicator: vernier calipers are used to measure the tumor volume twice a week and measure the long diameter and the short diameter of the tumor. A volume calculation formula thereof is as follows: volume=0.5×long diameter×short diameter²; and a relationship between the change of the tumor-bearing mouse tumor volume and the administration time is recorded, and experimental results are shown in FIG. 6.

It is shown through data in FIG. 6 that compared with the vehicle control group, the administration group has the ability to inhibit the tumor growth, and compared with the administration group rIL-10, with the prolongation of time, the administration group IL-10-Fc-A, the administration group IL-10-Fc-B, the administration group IL-10-Fc-D and the administration group IL-10-Fc-G have the stronger inhibitory effects on the tumor growth, and the inhibitory effects on the tumor growth are apparently better than the human interleukin-10, herein the fusion protein IL-10-Fc-A has the best inhibitory effect on the tumor growth.

### Example 6

This example is used to describe an in vivo efficacy experiment of a fusion protein of the present disclosure in a humanized xenografted non-small cell lung cancer H1975 tumor model.

1. Experimental animal:
Species strain: MusMusculus, NCG, mouse
Week age: 6-8 weeks
Body weight: 18-22 g
Number of animals: 32
Experimental animal provider: Jiangsu JicuiYaokang Biotechnology Co., Ltd.
   2. Cell culture: tumor cells (purchased from ATCC, the article number is CRL-5908) are cultured in an incubator at 37° C. and 5% CO₂ with a RPMI-1640 medium containing inactivated 10% FBS, 100 U/ml penicillin, 100 μg/ml streptomycin and 2 mM glutamine, the cells are passaged in bottles every 3 to 4 days after the cells are fully grown, and the tumor cells in a logarithmic growth phase are used for inoculation of a tumor in vivo.
   3. Inoculation and grouping of tumor cells:

The H1975 cells are washed twice with PBS, and then the tumor cells are resuspended in PBS, to obtain NCl-H1975 human non-small cell lung cancer cells, and the NCl-H1975 human non-small cell lung cancer cells are inoculated into a right lateral thorax portion of an experimental animal subcutaneously at 100 μl/mouse, and the inoculation amount of cells is 5×10⁶/mouse; and PBMC is derived from normal human peripheral blood, and is inoculated into a tumor-bearing mouse in three days before NCl-H1975 cell inoculation at 2×10⁶/mouse. While the tumor grows to about 50-80 mm³, it is administered in groups at a total of 4 groups, there are 8 animals in each group, and a specific dosing scheme is shown below in Table 3.

TABLE 3

| Group | Number of | | | | Administration | Administration |
|---|---|---|---|---|---|---|
| number | animals | Treatment | Dose | | route | cycle |
| 1 | 8 | Vehicle | — | | Subcutaneous | twice a week, |
| | | control group | | | injection | 6 doses |
| 2 | 8 | Fusion protein | 1.0 | mg/kg | Subcutaneous | twice a week, |
| | | IL-10-Fc-A | | | injection | 6 doses |
| 3 | 8 | Fusion protein | 0.1 | mg/kg | Subcutaneous | twice a week, |
| | | IL-10-Fc-A | | | injection | 6 doses |
| 4 | 8 | Fusion protein | 0.01 | mg/kg | Subcutaneous | twice a week, |
| | | IL-10-Fc-A | | | injection | 6 doses |

4. Detection indicator:
a. Tumor volume: vernier calipers are used to measure the tumor volume twice a week and measure the long diameter and the short diameter of the tumor, and a volume calculation formula thereof is as follows: volume=0.5×long diameter×short diameter².
b. Animal response after dosing: while the tumor volume is measured, the body weight of a mouse is weighed. A relationship between the change of the mouse body weight and the administration time Is recorded. At the same time, the survival situation and health status of the mouse, such as animal activity and feeding during an administration period, are observed.
c. Photographing of tumor body: at the end of the experiment, the mouse is euthanized, the tumor is removed, and the removed tumors of the control group and the test group are neatly arranged and photographed.
   5. Drug evaluation index:
a. Tumor growth inhibition rate (%)

Tumor growth inhibition rate (%)=(1−T/C)×100%

T/C=treatment group RTV/control group RTV

The tumor growth inhibition rate is ≥ 60%, and it is effective while statistically processed p is <0.05.
b. Tumor volume ratio T/C (%) of treatment group/control group

Tumor volume ratio T/C (%) of treatment group/control group=treatment group RTV/control group RTV×100%
Efficacy evaluation criteria: T/C (%)>40% is Invalid. T/C (%)≤40% and p<0.05 is effective.
   6. Immunological evaluation indicator: the blood is collected at the time of experimental grouping (within 3 days after grouping) and 24 hours after the last administration (the end of the experiment), PBMC is separated, and human CD45 is detected; and at the end of the experiment, the tumors are collected, cell suspension is prepared, and CD3/CD8/INF-r/CD4/CD25/FOXP3/PD-1/LAG3 is detected.
   7. Statistical analysis

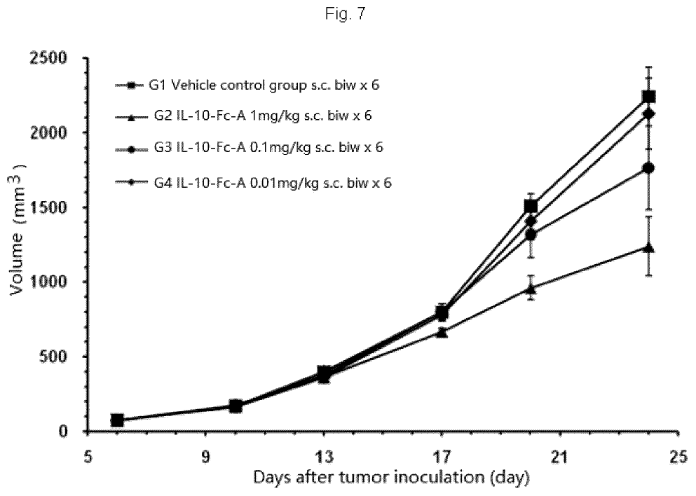

A One-Way ANOVA test is used for statistical analysis of the tumor volume between groups. and P<0.05 is considered to have a significant difference. Experimental results are shown in FIG. 7.

As shown in FIG. 7, compared with the vehicle control group, the administration group has the ability to inhibit the growth of the tumor, and the inhibitory ability of the different administration doses on the tumor shows a significant dose-effect relationship. The administration dose of the fusion protein IL-10-Fc-A is higher, the inhibitory ability to the tumor is stronger, and it still has the inhibitory ability to the tumor while the administration dose is 0.01 mg/kg. Therefore, it may be proved that the fusion protein IL-10-Fc-A provided by the present disclosure may be used to treat the tumor diseases.

The present disclosure is not limited to the above specific embodiments, and any persons may obtain other various forms of products under the inspiration of the present disclosure, but no matter if any changes are made in its shape or structure, all products with the same or similar technical schemes as the present disclosure fall within a scope of protection of the present disclosure.

What is claimed is:

1. A fusion protein containing a human interleukin-10 and an Fc fragment, wherein the fusion protein is formed by fusing the human interleukin-10 with the Fc fragment of an immunoglobulin IgG via a linker peptide, wherein, the Fc fragment is an Fc fragment of human IgG1, an Fc fragment of human IgG2 or an Fc fragment of human IgG4;
the Fc fragment of the human IgG4 is the amino acid sequence shown in SEQ ID No: 6; and
the human interleukin-10 is the amino acid sequence shown in SEQ ID No: 1.

2. (canceled)

3. The fusion protein according to claim 1, wherein the Fc fragment is the amino acid sequence shown in SEQ ID No: 2 or SEQ ID No: 3.

4. The fusion protein according to claim 1, wherein a general formula of the linker peptide is [GlyGlyGlyGlyX]ₙ;
wherein, X is Ser or Ala, and n is an integer of 1-6.

5. The fusion protein according to claim 4, wherein n is 6.

6. A polynucleotide, wherein the polynucleotide encodes the fusion protein according to claim 1.

7. A recombinant deoxyribonucleic acid (DNA) expression vector, wherein the recombinant DNA expression vector contains the polynucleotide according to claim 6.

8. A host cell for transfected with the recombinant DNA expression vector according to claim 7, wherein the host cell comprises at least one of a prokaryotic cell, a yeast cell and a mammalian cell.

9. A drug or a pharmaceutical composition, wherein the drug or the pharmaceutical composition comprises the fusion protein according to claim 1.

10. A method for preventing and/or treating an immune disease and/or a cancer, comprising administering effective amount of the fusion protein according to claim 1.


SEQUENCE LISTING (16 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 227 aa, Homo sapiens
DKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, Homo sapiens
EPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 30 aa, Homo sapiens
GGGGSGGGGSGGGGSGGGGSGGGGSGGGGS

SEQ ID NO: 5 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 6 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 25 aa, Artificial Sequence
  Synthesized
GGGGSGGGGSGGGGSGGGGSGGGGS

SEQ ID NO: 8 - protein, 20 aa, Artificial Sequence
  Synthesized
GGGGAGGGGAGGGGAGGGGA

SEQ ID NO: 9 - protein, 15 aa, Artificial Sequence
  Synthesized
GGGGSGGGGSGGGGS

SEQ ID NO: 10 - protein, 15 aa, Artificial Sequence
  Synthesized
GGGGAGGGGAGGGGA

SEQ ID NO: 11 - protein, 5 aa, Artificial Sequence
  Synthesized
  MISC_FEATURE (5)..(5): Xaa is Ser or Ala
GGGGX

SEQ ID NO: 12 - protein, 10 aa, Artifical sequence
  Synthesized
  MISC_FEATURE (5)..(5): Xaa is Ser or Ala
  MISC_FEATURE (10)..(10): Xaa is Ser or Ala
GGGGXGGGGX

SEQ ID NO: 13 - protein, 15 aa, Artificial Sequence
  Synthesized
  MISC_FEATURE (5)..(5): Xaa is Ser or Ala
  MISC_FEATURE (10)..(10): Xaa is Ser or Ala
  MISC_FEATURE (15)..(15): Xaa is Ser or Ala
GGGGXGGGGXGGGGX

SEQ ID NO: 14 - protein, 20 aa, Artificial Sequence
  Synthesized
  MISC_FEATURE (5)..(5): Xaa is Ser or Ala
  MISC_FEATURE (10)..(10): Xaa is Ser or Ala
  MISC_FEATURE (15)..(15): Xaa is Ser or Ala
  MISC_FEATURE (20)..(20): Xaa is Ser or Ala
GGGGXGGGGXGGGGXGGGGX

SEQ ID NO: 15 - protein, 25 aa, Artificial Sequence
  Synthesized
  MISC_FEATURE (5)..(5): Xaa is Ser or Ala
  MISC_FEATURE (10)..(10): Xaa is Ser or Ala
  MISC_FEATURE (15)..(15): Xaa is Ser or Ala
  MISC_FEATURE (20)..(20): Xaa is Ser or Ala
  MISC_FEATURE (25)..(25): Xaa is Ser or Ala
GGGGXGGGGXGGGGXGGGGXGGGGX

SEQ ID NO: 16 - protein, 30 aa, Artificial Sequence
  Synthesized
  MISC_FEATURE (5)..(5): Xaa is Ser or Ala
  MISC_FEATURE (10)..(10): Xaa is Ser or Ala
  MISC_FEATURE (15)..(15): Xaa is Ser or Ala
  MISC_FEATURE (20)..(20): Xaa is Ser or Ala
  MISC_FEATURE (25)..(25): Xaa is Ser or Ala
  MISC_FEATURE (30)..(30): Xaa is Ser or Ala
GGGGXGGGGXGGGGXGGGGXGGGGXGGGGX